(19) United States
(12) Patent Application Publication
(10) Pub. No.: US 2006/0029530 A1
(43) Pub. Date: Feb. 9, 2006

(54) Perfect tooth brush sterilize

(76) Inventor: Gloria Gremillion, Portallen, LA (US)

Correspondence Address: Gloria Gremillion, 650 California St., Port Allen, LA, 70767, US

(21) Appl. No.: 10/913,831

(22) Filed: Aug. 9, 2004

(51) Int. Cl. A61L 2/18

(52) U.S. Cl. 422/292; 206/209.1

(57) ABSTRACT

The Perfect Tooth Brush Sterilize is to put your tooth brush. So it can be free from living gern. Put the Canister half full of Antiseptic Mouth Rinse in the Perfect Tooth Brush Sterilize and your Tooth Brush will be free of gern. Best to make it from glass. So you can see to clean it. Clean it at lease once a month. The best color would be clear. And the best size is 12½ inches tall. The top would be pull up and push down. The best measure of the open for the top is (mm) 38 or 43 open. Very safe, no small parts.

Here is my Cross-Reference check;
Aug. 2, 1949 C. J. LUNDGREN Des, 154,689
Jul. 29, 1980 Billy G. Winston 4,214,657
Apr. 3, 1984 Rene Vignand 4,440,835
Sep. 25, 1984 Arthur Jump, JR. 4,473,152
Nov. 25, 1986 James O, Murdock 4,625,119
Jul. 26, 1988 Lucille Phillips 4,759,383
Feb. 7, 1989 Charles H. Ritter 4,803,364
Mar. 28, 1989 Mark R. Dusbabeck 4,816,648
Dec. 19, 1989 Charles H. Ritter 4,888,487
Aug. 21, 1990 Charles H. Ritter 4,950,902
Nov. 27, 1990 Wayne Lackey et al. 4,973,847
Jun. 11, 1991 Robert Foster, Jr. et al. 5,023,460
Sep. 10, 1991 Wayne Lackey, et al 319,875
Dec. 31, 1991 Eugene D. Stabe 322,732
Feb. 11, 1992 Ruben L. Gray 5,086,916
Feb. 9, 1993 John M. Zabsky et al. 5,185,532
Apr. 26, 1994 Eugene M. Coffey 5,306,176
Apr. 4, 1995 Elba Torres et al 356,915
Sep. 5, 1995 Robert M. Williams 5,447,684
Aug. 26, 1997 Willian Tooma 5,660,285
Mar. 17, 1998 Chung-jeng Pai 5,727,273
Dec. 29, 1998 Ronald H. winston 5,853,290
Jun. 29, 1999 Dale M. Frazell 5,915,868
Mar. 16, 1999 Lovelace Gipson 5,882,613
Sep. 14, 1999 Chia-Ping Lin 413,986
Oct. 5, 1999 Carel Vermooten et al. 5,960,801
Aug. 1, 2000 Melvin W. Peifer 6,096,264
Mar. 26, 2002 Robert Smith et al. 6,360,884 BI
May 20, 2003 Jose D. Herrera 6,565,819 BI
Aug. 5, 2003 David Naredo 6,601,699 BI

The Perfect Tooth Brush Sterilize is to put your tooth brush so it can be free from living gern.

Put the Canister half full of Antiseptic Mouth Rinse in the Perfect Tooth Brush Sterilize and your Tooth Brush will be free of gern

Best to make it from glass.

So you can see to clean it.

The best color would be clear.

And the best size is 12½ inches tall

The top would be pull up and push down.

The best measure of the open for the top is (mm) 38 or 43 open

### BRIEF SUMMARY OF THE INVENTION

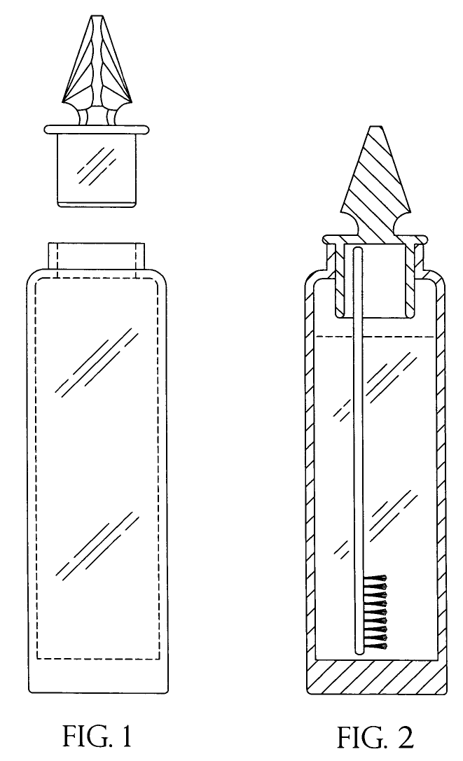

FIG. 1 is a whole view of a Perfect Tooth brush Sterilizer showing my new design;

FIG. 2 is a That show that it is clear glass and on each side that it is solid glass and botton is heavy glass and that the tooth brush go in the top of the tooth brush Sterilizer. The top that made with groove to make it easy to pick up and down. Tip top is solid glass. Then it open for the tooth brush to be right at your finger tip.

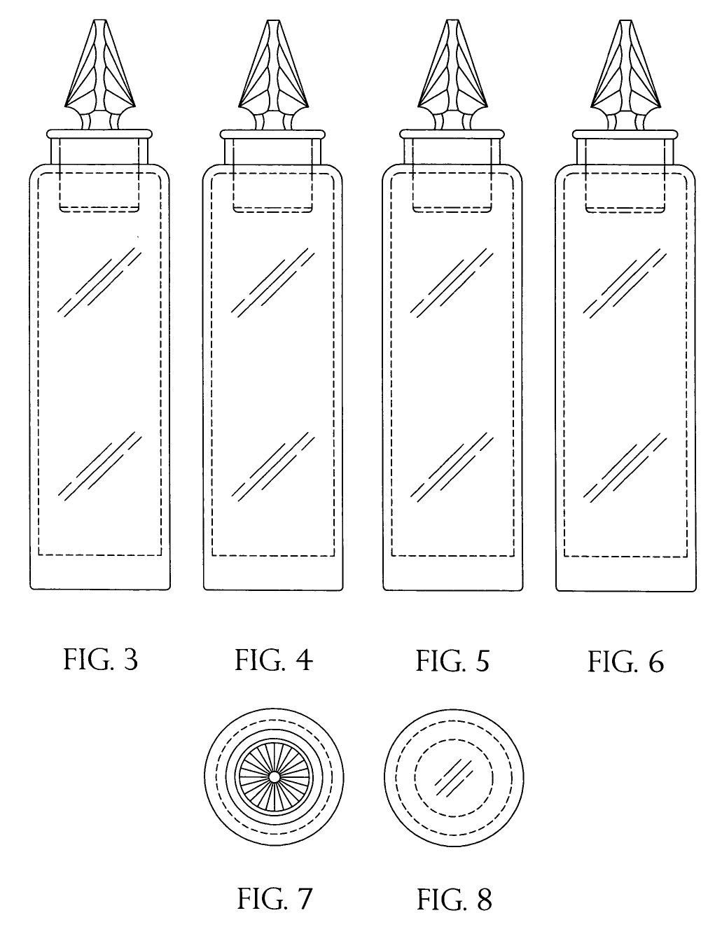

FIG. 3 is a front side view thereof.

FIG. 4 is a rear side view thereof;

FIG. 5 is a left side view thereof;

FIG. 6 is a right side view thereof;

FIG. 7 is a top view thereof;

FIG. 8 is a bottom view thereof;

I claim: The Utility design for a Perfect Tooth Brush Sterilizer as shown.

1. What I claim as my Invention is the Perfect Tooth Brush Sterilize. I claim the Perfect Tooth Brush Sterilize followed by the recitation of the particular matter which you regard as your invention:
1) The easy grip top to open sand close.
2) The Trop not to tight and not to loose where beta would go in.
3) Easy open to clean with a Sterilize brush.
4) Easy to hold in hand to clean.
5) Easy to put the gern Antiseptic in.
6) The thick heavy body so it will nest tilt over.
7) Anyone can use the Perfect Tooth Brush Sterilize.
8) No, small part for any one to put in mouth.
